(19) United States
(12) Patent Application Publication
(10) Pub. No.: US 2020/0380880 A1
(43) Pub. Date: Dec. 3, 2020

(54) KNOW-HOW INFORMATION PROCESSING SYSTEM, METHOD AND DEVICE

(71) Applicant: HONDA MOTOR CO., LTD., MINATO-KU, TOKYO (JP)

(72) Inventors: Atsushi NISHI, WAKO-SHI, SAITAMA-KEN (JP); Miyuki SHIMOTA, WAKO-SHI, SAITAMA-KEN (JP); Takaichi SANO, WAKO-SHI, SAITAMA-KEN (JP); Makoto YUZAWA, WAKO-SHI, SAITAMA-KEN (JP); Seiichi YAMAMOTO, MINATO-KU, TOKYO (JP); Keiichi IGUCHI, MINATO-KU, TOKYO (JP)

(21) Appl. No.: 16/617,927

(22) PCT Filed: May 29, 2018
(86) PCT No.: PCT/JP2018/020462

§ 371 Date: Nov. 27, 2019

(30) Foreign Application Priority Data
May 31, 2017 (JP) 2017-107344

(51) Int. Cl. G09B 5/12; G06F 3/01; A61B 5/16; G09B 19/00; A01G 7/00

(52) U.S. Cl. CPC G09B 5/125; G06F 3/011; G06Q 50/02; G09B 19/00; A01G 7/00; A61B 5/165

(57) ABSTRACT

Provided are a know-how information processing system, method and device with which it is possible to utilize advanced know-how which responds to changes in the surrounding environment. This know-how information processing system links action information and event information acquired at the same time or within a prescribed time period, records the same as a life log of a subject (operator), and outputs information in which the life log and environment information are associated with one another (know-how data) or information generated from the associated information (know-how content), as know-how information.

### TECHNICAL FIELD

The present invention relates to a know-how information processing system, method, and device adapted to process know-how information.

### BACKGROUND ART

In recent years, Information and Communication Technology (ICT) has been used in industries and business fields that tend to rely on implicit knowledge or rules of thumb (for example, agriculture, forestry and fisheries, and industrial arts manufacturing), and efforts to improve efficiency are progressing.

For example, in Japanese Laid-Open Patent Publication No. 2013-215099, a system is proposed in which plan data indicative of a production plan for a living organism is stored in advance, and by inputting the results of on-site operations performed in accordance with the production plan and production results (i.e., execution results), the system is configured to be capable of feeding back such information to a subsequent production plan. Consequently, a disclosure is provided to the effect that implicit knowledge (superior operational know-how) of an exemplary farmer (outstanding farmer) can be registered in a database as formalized information (difference data between the production plan and the execution results).

### SUMMARY OF INVENTION

Incidentally, assuming that this type of operational know-how includes knowledge that directly and immediately influences the production results, even if there are slight differences, knowledge is also provided that can influence the production results when viewed from a broad perspective. As examples of the latter, there may be cited the sensation of slight changes in the surrounding environment, and making on-the-fly changes to the content of operations as circumstances demand. By accumulating such advanced know-how (skills of a worker) in the form of formal knowledge, it can be expected to lead to further improvements in productivity.

However, with the system proposed in Japanese Laid-Open Patent Publication No. 2013-215099, an operator must perform a predetermined input operation in order to input the execution results. Stated otherwise, in the case that an operator takes an action for which the immediate effectiveness with respect to the production results is low, for example, in the case that an action is taken that does not directly lead to an improvement in the production results or the production efficiency, unless the worker recognizes that such an action is useful, a problem results in that the input and accumulation of know-how that is substantiated by such an action cannot be realized.

The present invention has been devised in order to solve the aforementioned problems, and has the object of providing a know-how information processing system, method, and device in which advanced know-how corresponding to changes in the surrounding environment can be put to practical use.

A know-how information processing system according to a first aspect of the present invention comprises an action information acquisition unit configured to acquire action information indicative of an action of a target person, an event information acquisition unit configured to acquire event information related to an event that occurs in vicinity of the target person or an event involving the target person, a life log recording unit configured to associate the action information acquired by the action information acquisition unit and the event information acquired by the event information acquisition unit, which are acquired respectively at the same time or within a predetermined time period, and record associated information as a life log of the target person, an environmental information acquisition unit configured to acquire environmental information indicative of a surrounding environment in the vicinity of the target person in which the action or the event occurs, and a know-how information output unit configured to output as know-how information associated information in which the life log recorded by the life log recording unit is associated with the environmental information acquired by the environmental information acquisition unit, or information generated from the associated information.

In the foregoing manner, the action information and the event information are associated with each other when the life log is recorded, and information associated with the environmental information or information generated from the associated information is output. Consequently, it is possible to put to practical use the information, and more specifically, the advanced know-how corresponding to changes in the surrounding environment, in which the action, the event, and the surrounding environment of the target person are mutually associated.

Further, the know-how information processing system may further comprise a biological information acquisition unit configured to acquire biological information indicative of a biological activity of the target person, and an emotional information acquisition unit configured to acquire emotional information indicative of an emotional state of the target person on a basis of the biological information acquired by the biological information acquisition unit, wherein the life log recording unit may be configured to associate the action information acquired by the action information acquisition unit, the event information acquired by the event information acquisition unit, and the emotional information acquired by the emotional information acquisition unit, which are acquired respectively at the same time or within the predetermined time period, and record associated information as the life log.

Further, the know-how information processing system may further comprise a life log specifying unit configured to specify as a notable log a life log in which a change in the emotional information is greater than or equal to a threshold value, wherein the know-how information output unit may be configured to output the know-how information including the notable log specified by the life log specifying unit. In accordance with such features, it becomes possible to detect an unconscious awareness of the target person through a change of the emotional state, and it is possible to appropriately extract the notable log which can serve as know-how from among a large number of the life logs.

Further, the know-how information processing system may further comprise a life log specifying unit configured to specify as a notable log, from among life logs recorded in past by the life log recording unit and which are associated with the events that are identical or similar to each other, a life log in which a degree of statistical divergence of the action information is greater than or equal to a threshold value, wherein the know-how information output unit may be configured to output the know-how information including the notable log specified by the life log specifying unit. In accordance with such features, it becomes possible to detect an unconscious awareness of the target person through the statistical divergence of the action information, and it is possible to appropriately extract the notable log which can serve as know-how from among a large number of the life logs.

Further, the know-how information processing system may further comprise an evaluation assignment unit configured to assign an evaluation result of success or failure to the action or the event, and a life log specifying unit configured to specify as a notable log a life log whose evaluation result assigned by the evaluation assignment unit has a degree of success or failure that is greater than a threshold value, wherein the know-how information output unit may be configured to output the know-how information including the notable log specified by the life log specifying unit. In accordance with such features, from among a large number of the life logs, it is possible to appropriately extract the notable log to which there is assigned a certain evaluation to the effect of being indicative of successful know-how or failure know-how.

Further, the know-how information output unit may be configured in a manner to generate a virtual world in which the action is virtually experienced using at least the notable log. In accordance with this feature, the experiences of the target person can be shared through the virtual world, and the know-how can be efficiently learned and acquired.

Further, the life log recording unit may record life logs for respective target persons, and the know-how information output unit may output the know-how information provided by a first target person to a second target person that differs from the first target person, and who has provided a life log having a combination of an event and a surrounding environment which is identical or similar to a combination of the event and the surrounding environment indicated by the know-how information. In accordance with such features, advanced know-how can be shared under a situation in which the combination of the events and the surrounding environment is closely related, and know-how can be utilized mutually between the target persons.

In a know-how information processing method according to a second aspect of the present invention, there are executed by one or a plurality of computers an action information acquisition step of acquiring action information indicative of an action of a target person, an event information acquisition step of acquiring event information related to an event that occurs in vicinity of the target person or an event involving the target person, a life log recording step of associating the action information and the event information, which are acquired respectively at same time or within a predetermined time period, and recording associated information as a life log of the target person, an environmental information acquisition step of acquiring environmental information indicative of a surrounding environment in the vicinity of the target person in which the action or the event occurs, and a know-how information outputting step of outputting as know-how information associated information in which the recorded life log is associated with the acquired environmental information, or information generated from the associated information.

In a know-how information processing device according to a third aspect of the present invention, at least a first computer, a second computer, and an environmental sensor group are connected via a network, wherein the first computer is configured to associate action information indicative of an action of the target person, and event information related to an event that occurs in vicinity of the target person or an event involving the target person, and record associated information as a life log of a target person, the environmental sensor group is configured to acquire environmental information indicative of a surrounding environment in the vicinity of the target person in which the action or the event occurs, and the second computer is configured to output as know-how information associated information in which the life log recorded by the first computer is associated with the environmental information acquired by the environmental sensor group, or information generated from the associated information.

According to the know-how information processing system, the method, and the device according to the present invention, it is possible to put to practical use the advanced know-how corresponding to changes in the surrounding environment.

### BRIEF DESCRIPTION OF DRAWINGS

FIG. 1 is a block diagram showing a configuration of a know-how information processing system according to an embodiment of the present invention;

FIG. 2 is a functional block diagram of a production management server shown in FIG. 1;

FIG. 3 is a first flowchart in which there is provided a description of operations of the know-how information processing system shown in FIG. 1;

FIG. 4 is a second flowchart in which there is provided a description of operations of the know-how information processing system shown in FIG. 1;

FIG. 5A is a diagram illustrating by example a data structure of a life log;

FIG. 5B is a diagram illustrating by example a data structure of environmental information;

FIG. 5C is a diagram illustrating by example a data structure of know-how data;

FIG. 6 is a diagram schematically illustrating a life log specifying method;

FIG. 7 is a third flowchart in which there is provided a description of operations of the know-how information processing system shown in FIG. 1;

FIG. 8A and FIG. 8B are diagrams illustrating by example a data structure of operation information;

FIG. 9 is a diagram illustrating an example of a form in which know-how content is output;

FIG. 10 is a diagram schematically showing a specifying method according to a first modification; and

FIG. 11 is a diagram schematically showing a specifying method according to a second modification.

### DESCRIPTION OF EMBODIMENTS

Hereinafter, a preferred embodiment of a know-how information processing system according to the present invention will be presented and described with reference to the accompanying drawings, in relation to a know-how information processing method and a know-how information processing device.

### [Configuration of Know-How Information Processing System 10]

### <Overall Configuration>

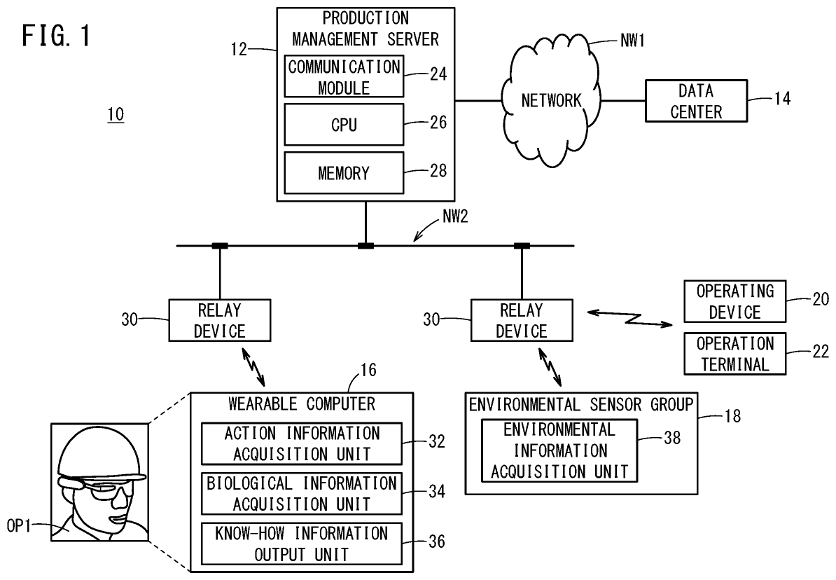

FIG. 1 is a block diagram showing a configuration of a know-how information processing system 10 according to an embodiment of the present invention. The know-how information processing system 10 is a system that manages the production of a product (for example, an agricultural product such as cabbage or the like), and is configured to be capable of accumulating in a database know-how information concerned with production activities.

The know-how information processing system 10 is configured to include a production management server 12, a data center 14, a wearable computer 16, an environmental sensor group 18, an operating device 20, and an operation terminal 22.

The production management server 12 is a computer (first computer) that comprehensively controls one or a plurality of such operating devices 20 provided at a production site. The production management server 12 is configured to include a communication module 24, a CPU 26 (Central Processing Unit), and a memory 28. Moreover, the memory 28 is configured in the form of a non-transitory and computer-readable storage medium.

A plurality of types of databases, which will be described later, are constructed by a server (not shown) installed in the data center 14. The data center 14 is configured to be capable of communicating with the production management server 12 via a network NW1 (the Internet). Consequently, data can be exchanged between the production management server 12 and the data center 14.

Incidentally, the production management server 12 and a plurality of relay devices 30, 30 are connected to each other via a network NW2 (an intranet). Consequently, the production management server 12 is capable of receiving data from the wearable computer 16 (second computer), the environmental sensor group 18, the operating device 20, or the operation terminal 22 via the relay devices 30 and the network NW2.

The wearable computer 16 is a multi-function/multi-purpose device which is usable in a state of being worn by an operator OP1 (first target person), and is configured to include, for example, a casing, a control substrate, a display panel, a speaker, or a plurality of sensors. Consequently, the wearable computer 16 functions as an action information acquisition unit 32, a biological information acquisition unit 34, and a know-how information output unit 36. More specifically, the various functional units of the action information acquisition unit 32, the biological information acquisition unit 34, and the know-how information output unit 36 are software based functional units, in which the various functions thereof are realized by the CPU executing programs stored in the main memory. However, the various functions thereof can also be realized by hardware based functional units made up from integrated circuits such as FPGAs (Field-Programmable Gate Arrays) or the like.

The sensors that are mounted on the wearable computer 16 may include, for example, any of a camera, a vital sensor, a motion sensor, a position sensor, or an audio sensor. By analyzing values from such sensors, it is possible to detect, for example, a heartbeat, a pulse, the blood pressure, the pupils, a line of sight, movements, a position, or the voice of the operator OP1.

The environmental sensor group 18 is a collection of sensors that measure the environment of the production site, and functions as an environmental information acquisition unit 38. The environmental sensors may include, for example, any of a thermometer, a hygrometer, an illumination meter, a barometer, a weight scale, or an ultraviolet actinometer. Further, a plurality of environmental sensors of the same type may be installed at a plurality of locations within the production site.

The operating device 20 is a peripheral device used at the production site, and more specifically, is a farming implement, an agricultural vehicle, or a tool. The operation terminal 22 is a terminal device (for example, a personal computer or a tablet) used for monitoring the state of the production site.

### <Functional Block Diagram of Production Management Server 12>

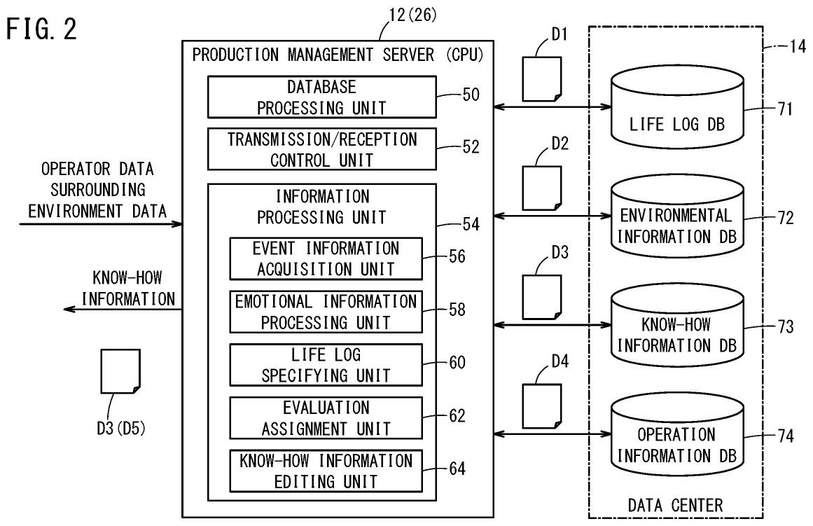

FIG. 2 is a functional block diagram of the production management server 12 shown in FIG. 1.

By reading out and executing programs stored in the memory 28, the CPU 26 of the production management server 12 functions as a database processing unit 50, a transmission/reception control unit 52, and an information processing unit 54. The information processing unit 54 includes an event information acquisition unit 56, an emotional information acquisition unit 58, a life log specifying unit 60, an evaluation assignment unit 62, and a know-how information editing unit 64.

More specifically, according to the present embodiment, the various functional units of the database processing unit 50, the transmission/reception control unit 52, and the information processing unit 54 are software based functional units, in which the various functions thereof are realized by the CPU 26 executing programs stored in the main memory. However, the various functions thereof can also be realized by hardware based functional units made up from integrated circuits such as FPGAs (Field-Programmable Gate Arrays) or the like.

The data center 14 includes, for example, four types of databases, and more specifically, a database related to life logs D1 (hereinafter referred to as a life log DB 71), a database related to environmental information D2 (hereinafter referred to as an environmental information DB 72), a database related to know-how data D3 (hereinafter referred to as a know-how information DB 73), and a database related to operation information D4 (hereinafter referred to as an operation information DB 74).

### [Operations of Know-How Information Processing System 10]

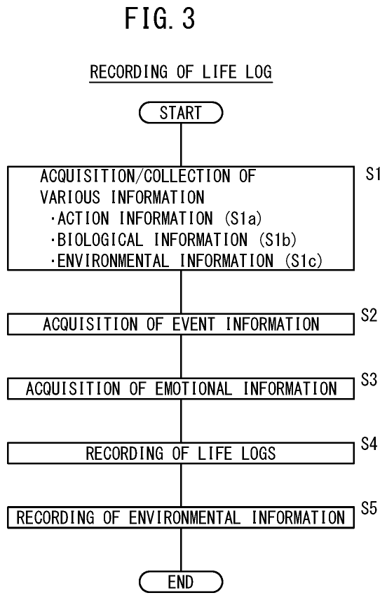

The know-how information processing system 10 according to the present embodiment is configured in the manner described above. Next, a description will be given with reference to FIGS. 3 to 9 concerning operations of the know-how information processing system 10.

### <First Operation; Recording of Life Logs D1>

In step S1 of FIG. 3, the know-how information processing system 10 acquires various types of information used for generating the life logs D1. More specifically, in step S1a, the action information acquisition unit 32 acquires information (hereinafter referred to as action information) indicative of actions of the operator OP1. In step S1b, the biological information acquisition unit 34 acquires information (hereinafter referred to as biological information) indicative of a biological activity of the operator OP1.

Moreover, steps S1a and S1b may be executed either synchronously or asynchronously (for example, at different periods). With the accumulation of a predetermined amount of data acting as a trigger, the wearable computer 16 transmits data in relation to the operator OP1 (hereinafter referred to as operator data), which includes the action information and the biological information, to the production management server 12.

On the other hand, in step S1c, the environmental information acquisition unit 38 acquires information (hereinafter referred to as environmental information) indicative of the surrounding environment in the vicinity of the operator OP1. With the accumulation of a predetermined amount of data acting as a trigger, the environmental sensor group 18 transmits data (hereinafter referred to as surrounding environment data), which includes the environmental information, to the production management server 12.

The production management server 12 receives and acquires various information (the operator data and the surrounding environment data) via the relay devices 30, the network NW2, and the communication module 24, and thereafter, temporarily stores such information in the memory 28.

In step S2, the event information acquisition unit 56 acquires event information which is associated with the information (in particular, the operator data) that was acquired in step S1. More specifically, the event information acquisition unit 56 acquires the event information by extracting a portion of the operator data, or alternatively, by analyzing the content of the operator data and estimating an event.

In this instance, the term “event information” implies information in relation to events that occur in the vicinity of the operator OP1, or events involving the operator OP1. Such events may be units of action which are classified to an extent whereby an analysis of the action is possible, or for example, may be the name of a production process (for example, process A or process B), or may be an individual or a specific action (for example, a movement, an action of transporting, or an observation).

In step S3, the emotional information acquisition unit 58 acquires emotional information which is associated with the information (in particular, the operator data) that was acquired in step S1. More specifically, the emotional information acquisition unit 58 estimates the emotional state of the operator OP1 on the basis of the biological information included in the operator data, and acquires the result (a quantitative value) of such an estimation as the emotional information.

Prior to preforming such an estimation, the emotional information acquisition unit 58 may capture, for example, a change in the heart rate to thereby detect an emotional arousal, or may detect a facial expression by performing image processing on a camera image including the face. In addition, on the basis of one or a plurality of the aforementioned detection results, the emotional information acquisition unit 58 quantifies the emotional state of the operator OP1 using one or a plurality of parameters (for example, happiness/anger/sadness/enjoyment).

In step S4, the data center 14 (and more specifically, the life log DB 71) associates the information (with the exception of the environmental information D2) that was acquired in steps S1 to S3, and records such information as the life log D1 of the operator OP1. Prior to such recording, the database processing unit 50 performs a process (i.e., a data combining process) of associating information acquired within the same period or within a predetermined time period.

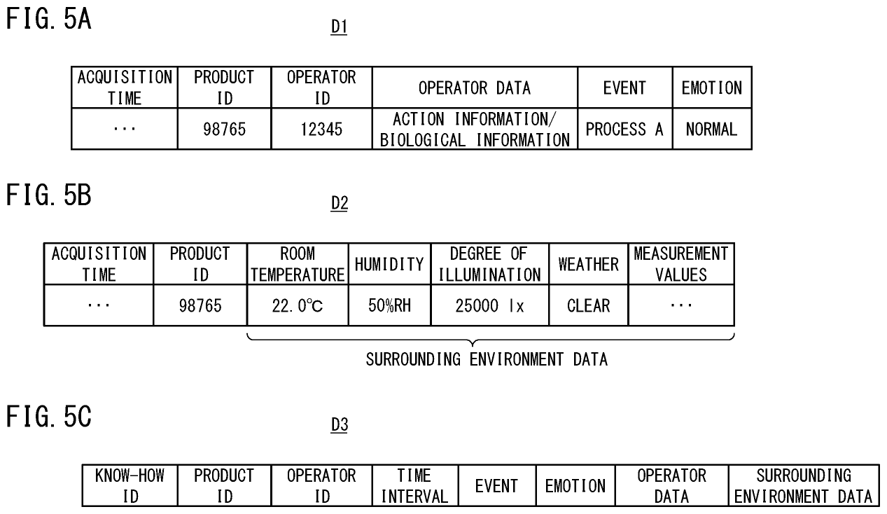

FIG. 5A is a diagram illustrating by example a data structure of a life log D1. The present drawing corresponds to a record which makes up a constituent unit of the life log DB 71. The life log D1 is configured to include an acquisition time, a product ID, an operator ID, operator data, an event, and an emotion. In this instance, the product ID (for example, “98765”) is an identifier unique to the product or a production lot, and the operator ID (for example, “12345”) is an identifier unique to the operator OP1.

Moreover, in the case that the record of the life log D1 shown in FIG. 5A is generated, a freely-selected type of database including, for example, a hierarchical type, a network type, or a relational type may be adopted. The same considerations apply to the environmental information D2 (FIG. 5B) and the know-how data D3 (FIG. 5C) and the like, which will be described later.

Thereafter, the transmission/reception control unit 52 transmits the data to be recorded including the life log D1 to the data center 14. Consequently, the life log DB 71 records the life log D1 of the operator OP1, which is created by associating at least the action information, the biological information, the event information, and the emotional information.

In step S5, the data center 14 (and more specifically, the environmental information DB 72) records the environmental information D2 that was acquired in step S1c. Prior to such recording, the transmission/reception control unit 52 transmits the data to be recorded including the environmental information D2 to the data center 14.

FIG. 5B is a diagram illustrating by example a data structure of the environmental information D2. The present drawing corresponds to a record which makes up a constituent unit of the environmental information DB 72. The environmental information D2 is configured to include an acquisition time, a product ID, and surrounding environment data (for example, the room temperature, the humidity, a degree of illumination, the weather, and other measurement values).

In this manner, the know-how information processing system 10 completes the first operation (recording of the life log D1). By repeatedly executing the flowchart of FIG. 3 in a periodical or irregular manner, the know-how information processing system 10 is capable of sequentially collecting life logs D1 of at least one target person who is involved with the production site.

### <Second Operation; Generation of Know-How Data D3>

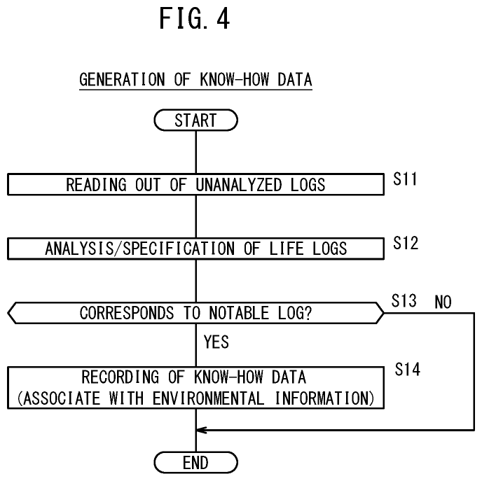

In step S11 of FIG. 4, the database processing unit 50 refers to the life log DB 71 of the data center 14, and reads out life logs D1 (a freely-selected number of records) that have not yet been subjected to analysis. The state of analysis is determined, for example, by referring to tag information (for example, a flag indicating the state of analysis) of the life logs D1.

In step S12, the life log specifying unit 60 analyzes the content of the life logs D1 that were read out in step S11, and specifies whether or not the life logs are capable of serving as know-how (hereinafter referred to as a notable log).

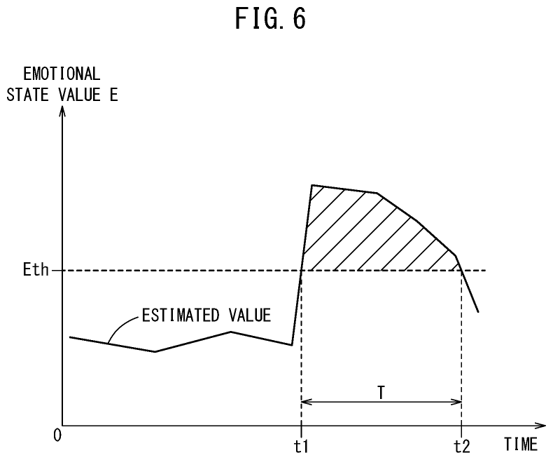

FIG. 6 is a diagram schematically illustrating a method of specifying the life logs D1. The horizontal axis of the graph indicates an acquisition time (hereinafter referred to simply as a “time t”) of the biological information, and the vertical axis of the graph indicates an emotional state value E (units: freely selected). The state value E is a parameter indicative of a degree of emotional arousal. In the case that the value of E is large, a state of emotional arousal is indicated, whereas in the case that the value of E is small, a normal state is indicated.

In this instance, the life log specifying unit 60 compares a magnitude relationship between a time-based change of the state value E and a preset threshold value Eth. As a result, it is considered that the state value E (estimated value) satisfies the relationship E≥Eth within the hatched region (i.e., within a time range T from time t1 to time t2). In this case, the life log specifying unit 60 specifies life logs D1 in which the value (t) of an “acquisition time” lies within a range of time t1≤T≤time t2.

In step S13, the life log specifying unit 60 determines whether or not the life logs D1 analyzed in step S12 correspond to a notable log. More specifically, in the case it is determined that a notable log does not exist among the targets of analysis (step S13: NO), the flowchart is brought to an end without any further processing. On the other hand, in the case it is determined that a notable log exists among the targets of analysis (step S13: YES), the process proceeds to the next step S14.

In step S14, the data center 14 (and more specifically, the know-how information DB 73) records the know-how data D3 including the notable log that was specified in step S13. Prior to such recording, the database processing unit 50 performs a process (i.e., a data combining process) of associating information acquired within the same period or within a predetermined time period.

In this instance, in the case that the acquisition time included in the life log D1 and the acquisition time included in the environmental information D2 coincide with each other within an allowable range (for example, within one hour), the database processing unit 50 combines the life log D1 and the environmental information D2 as being related to each other. Moreover, in the case there are a plurality of instances of the environmental information D2 in which the acquisition locations are different and the acquisition times coincide, the environmental information D2 which is acquired at a position closest to the operator can be used.

FIG. 5C is a diagram illustrating by example a data structure of the know-how data D3. The present drawing corresponds to a record which makes up a constituent unit of the know-how information DB 73. The know-how data D3 is configured to include a know-how ID, a product ID, an operator ID, a time interval, an event, an emotion, operator data, and surrounding environment data. In this instance, the know-how ID is an identifier unique to the know-how data D3, which is managed in a centralized manner by the know-how information DB 73.

Thereafter, the transmission/reception control unit 52 transmits the data to be recorded including the know-how data D3 to the data center 14. Consequently, the know-how information DB 73 records the know-how data D3 caused by the operator OP1.

In this manner, the know-how information processing system 10 completes the second operation (generation of the know-how data D3). By repeatedly executing the flowchart of FIG. 4, for example, during a time period in which the operating ratio of the production management server 12 is low, the know-how information processing system 10 is capable of generating the know-how data D3 from a large number of life logs D1.

### <Third Operation; Outputting of the Know-How Information>

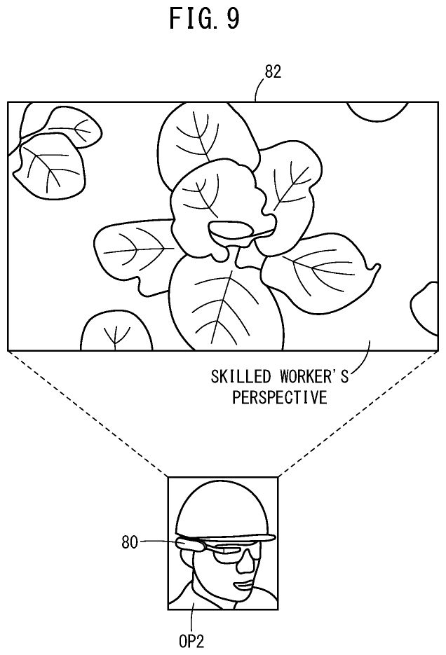

Incidentally, there may be a situation in which an operator OP2 (a second target person, refer to FIG. 9) having a low level of skill desires to proceed with the operation of “Process A”, by referring to the “skill of a worker” of the operator OP1 (see FIG. 1) who possesses a high level of skill. In this case, the know-how information processing system 10 is capable of providing know-how content D5 with respect to the operator OP2.

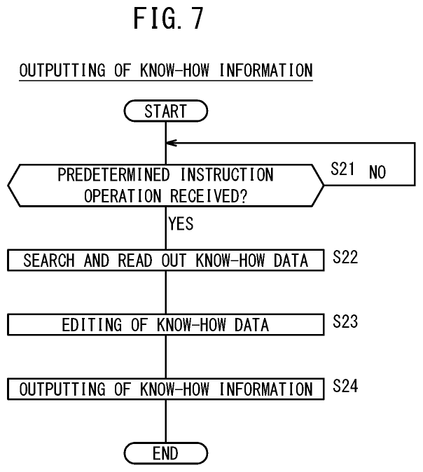

In step S21 of FIG. 7, the production management server 12 determines whether or not a predetermined instruction operation from an external device (for example, a wearable computer 80, see FIG. 9) has been received. As an interface (HMI; Human Machine Interface) for accepting the instruction operation, various forms, including a form selected from among a displayed content list, may be adopted.

In the case that an instruction operation has not yet been received (step S21: NO), the process remains at step S21 until such an operation is received. On the other hand, in the case that an instruction operation has been received (step S21: YES), the process proceeds to the next step S22.

In step S22, the database processing unit 50 performs a search process of the know-how information DB 73 in accordance with the instruction (selection of content) made in step S21, and reads out know-how data D3 which matches the search condition. As an exemplary search condition, there may be cited, for example, that a combination of an event and the surrounding environment is identical or similar.

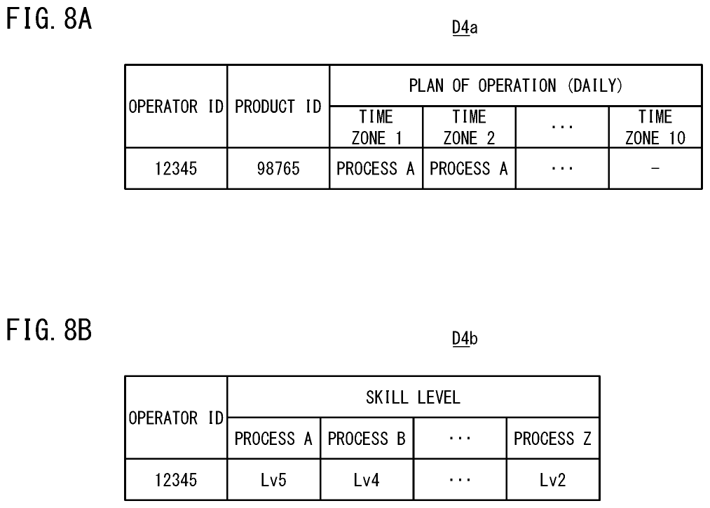

As shown in FIG. 8A, the operation information D4a includes an operator ID, a product ID, and a daily plan of operations. Within such a plan of operations, event information (in this instance, names of production processes) corresponding to a plurality of time zones (ten individual segments) are stored.

As shown in FIG. 8B, the operation information D4b includes an operator ID and skill levels for each of respective events (in this instance, for each process). Within the skill levels, there are stored levels of skill corresponding respectively to a plurality of events (more specifically, any one of five stage levels from Lv1 to Lv5).

For example, storage of the skill levels in the operation information D4b is carried out in the following manner.

(a) At a first time (before initiating the collection of life logs), when an electronic questionnaire (confirmation of work experience and skills) is answered, a level of skill is automatically calculated and stored based on the content of the answers.

(b) After having initiated the collection of life logs, the level of skill (after having been stored) is updated based on the number of years after having initiated the collection of life logs, and skills estimated from the life logs (images and actions).

For example, by the database processing unit 50 referring to the operation information D4a and D4b that is stored in the operation information DB 74, a product ID (for example “98765”) which satisfies the conditions of “event=process A” and “skill level=Lv5” can be specified.

In step S23, as needed, the know-how information editing unit 64 performs an editing process on the know-how data D3 that was read out in step S22. As exemplary editing processes, there may be cited, for example, [1] an annotation process of adding annotations to a moving image or a still image, [2] a graph creation process of presenting time-series data in the form of a graph, and [3] a thinning process or a trimming process of shortening the playback time of a moving image.

Hereinafter, the know-how data D3 and the know-how content D5 may be collectively referred to as “know-how information”. Moreover, the edited know-how content D5 may be recorded in the know-how information DB 73.

In step S24, the know-how information output unit 36 outputs to the operator OP2 the know-how information that was obtained in step S22 or step S23. Prior to being output in this manner, the transmission/reception control unit 52 transmits the know-how information to the wearable computer 80. The wearable computer 80 visually displays the know-how information (in this instance, the know-how content D5) using a display function.

As shown in FIG. 9, the operator OP2 wears a wearable computer 80 having the same device configuration as in the case of FIG. 1. In a display area of the wearable computer 80, a virtual world 82 (in this instance, cabbage as an agricultural product) is generated in which an action is projected from the viewpoint of the operator OP1.

In the foregoing manner, the know-how information output unit 36 may be configured in a manner to generate the virtual world 82 in which the action of the operator OP1 is virtually experienced using at least the notable log. In accordance with this feature, the experiences of the operator OP1 can be shared through the virtual world 82, and the know-how can be efficiently learned and acquired.

Further, the life log DB 71 is capable of recording life logs D1 for respective target persons. The know-how information output unit 36 may output the know-how information provided by the operator OP1 (i.e., the know-how data D3 or the know-how content D5) to an operator OP2 who differs from the operator OP1. Moreover, the operator OP2, for example, is an operator who has provided a life log D1 having a combination of the events and the surrounding environment which is identical or similar to a combination of the events and the surrounding environment indicated by the know-how information provided by the operator OP1. In accordance with such features, advanced know-how can be shared under a situation in which the combination of the events and the surrounding environment is closely related, and the know-how can be utilized mutually.

### <Analysis of Know-How Information>

A manufacturer may make use of the know-how information (successful experiences) for which a skilled worker (the operator OP1) has expressed feelings of “happiness” or “enjoyment”, and for each of events, may analyze the know-how information (including ordinary feelings) obtained in a process leading to a successful experience. For example, when “happiness” is indicated in a “harvest” event, it is possible to decipher from the know-how information the kind of operation that was performed with respect to which type of weather. By determining beforehand standard operations commensurate with weather patterns, an operator OP2 having a low level of skill can perform an operation that is suitable in relation to the weather situation he or she is confronting.

Similarly, a manufacturer may make use of the know-how information (a problem case) for which a skilled worker (the operator OP1) has expressed feelings of “anger” or “sadness”, and for each of events, may analyze the know-how information (including ordinary feelings) obtained in a process leading to a problem case, or obtained after the occurrence of such a problem case. For example, it is possible to decipher from the know-how information the factors (for example, bad weather) that caused or brought about the problem case, and how a skilled worker dealt with such a problem case (for example, by carrying out harvesting faster than usual, changing a fertilizer, etc.). By determining beforehand standard operations commensurate with problem patterns, an operator OP2 having a low level of skill can perform an operation that is suitable in relation to a crisis he or she is confronting.

Further, the method of utilizing the know-how information obtained from a skilled worker (the operator OP1) is not limited to a case of providing the information to an operator OP2 that differs from the skilled worker, and for example, may be utilized in relation to an automated control of the operating device 20. As a specific example thereof, the operating device 20 may be made to learn an amount and a timing for application of a fertilizer corresponding to a level of maturity of a crop, or a timing of watering or harvesting the crop in accordance with weather conditions, and such learning may be reflected in an automated operation such as application of the fertilizer or watering, or reaping or harvesting a crop by the operating device 20.

[Advantages and Effects in Accordance with the Know-How Information Processing System 10]

As described above, the know-how information processing system 10 is equipped with [1] the action information acquisition unit 32 configured to acquire the action information indicative of an action of a target person (the operator OP1 or OP2), [2] the event information acquisition unit 56 configured to acquire the event information related to an event that occurs in the vicinity of the target person or an event involving the target person, [3] the life log recording unit (life log DB 71) configured to associate the action information and the event information, which are acquired respectively at the same time or within a predetermined time period, and record the associated information as a life log D1 of the target person, [4] the environmental information acquisition unit 38 configured to acquire the environmental information D2 indicative of the surrounding environment in the vicinity of the target person in which the action or the event occurs, and [5] the know-how information output unit 36 configured to output as the know-how information the associated information (know-how data D3) in which the recorded life log D1 is associated with the acquired environmental information D2, or the information (the know-how content D5) generated from the know-how data D3.

Further, according to the know-how information processing method and device, there are executed by one or a plurality of computers [1] the action information acquisition step (step S1a) of acquiring the action information, [2] the event information acquisition step (step S2) of acquiring the event information, [3] the recording step (step S4) of recording the life log D1, [4] the environmental information acquisition step (step S1c) of acquiring the environmental information D2, and [5] the outputting step (step S24) of outputting as the know-how information the know-how data D3 in which the life log D1 and the environmental information D2 are associated, or the know-how content D5 generated from the know-how data D3.

In accordance with these features, since the action information and the event information are associated when the life log D1 is recorded, and further, the know-how data D3 associated with the environmental information D2 or the know-how content D5 is output, it is possible to put to practical use the information, and more specifically, the advanced know-how corresponding to changes in the surrounding environment, in which the actions, the events, and the surrounding environment of the target person are mutually associated.

Further, the know-how information processing system 10 may further be equipped with [6] the biological information acquisition unit 34 configured to acquire the biological information indicative of a biological activity of the target person, and [7] the emotional information acquisition unit 58 configured to acquire the emotional information indicative of an emotional state of the target person on the basis of the acquired biological information, wherein [8] the life log DB 71 may be configured to associate the action information, the event information, and the emotional information, which are acquired respectively at the same time or within the predetermined time period, and record associated information as the life log D1.

Further, the know-how information processing system 10 may further comprise [9] the life log specifying unit 60 configured to specify as a notable log the life log D1 in which a change in the emotional information is greater than or equal to a threshold value, wherein [10] the know-how information output unit 36 may be configured to output the know-how information (i.e., the know-how data D3 or the know-how content D5) including the notable log that was specified.

In accordance with such features, it becomes possible to detect an unconscious awareness of the operator through a change of the emotional state, and it is possible to appropriately extract the notable log which can serve as know-how from among a large number of the life logs D1.

### [Modifications (Specification of Life Log D1)]

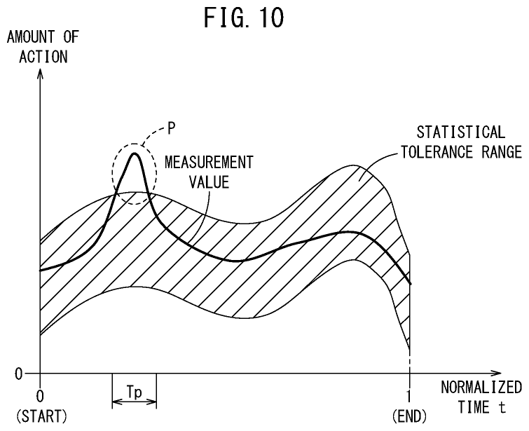

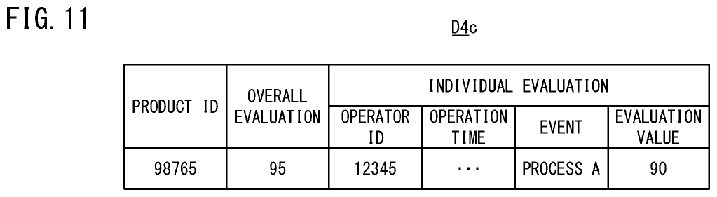

Next, a description will be given with reference to FIGS. 10 and 11 concerning modifications (in this instance, step S12 of FIG. 4) of the know-how information processing system 10.

### <Modification 1>

FIG. 10 is a diagram schematically showing a specifying method according to a first modification. The horizontal axis of the graph indicates time which has been normalized to within a range of [0, 1] (hereinafter referred to simply as “normalized time”), and the vertical axis of the graph indicates the amount of action (units: freely selected). In the case that the value of the amount of action is large, the target person undertakes a large action, whereas in the case that the value of the amount of action is small, the target person undertakes a small action.

In this instance, the life log specifying unit 60 determines whether or not a time-based change of the action information lies within a statistical tolerance range. As a result, it is assumed that the amount of action (measurement value) deviates from the statistical tolerance range within a time period range P that is surrounded by the dashed line. In this case, the life log specifying unit 60 specifies the life log D1 that lies within the range of the entire interval [0, 1].

Moreover, the statistical tolerance range is a range determined in accordance with past life logs D1 which are associated with events that are identical or similar to each other. More specifically, the statistical tolerance range is formed by adding a margin amount that is equivalent in a positive direction and a negative direction with respect to an average function of the life log D1.

In this manner, the life log specifying unit 60 may be configured to specify as a notable log, from among life logs D1 recorded in the past by the life log DB 71 and which are associated with the events that are identical or similar to each other, a life log D1 in which the degree of statistical divergence of the action information is greater than or equal to a threshold value. Consequently, it becomes possible to detect an unconscious awareness of the target person through the statistical divergence of the action information, and it is possible to appropriately extract the notable log which can serve as know-how from among a large number of the life logs D1.

### <Modification 2>

The life log specifying unit 60 may specify as a notable log a life log D1 to which there is assigned an evaluation result having a degree of success or failure that is greater than a threshold value. Prior to making such a specification, the evaluation assignment unit 62 assigns an evaluation result of success or failure with respect to actions or events after production is completed, or alternatively, during production.

In addition, the transmission/reception control unit 52 transmits the data to be recorded including the evaluation result to the data center 14. Consequently, the operation information DB 74 overwrites and updates a portion of the operation information D4c (see FIG. 11).

As shown in FIG. 11, the operation information D4c is configured to include a product ID, an overall evaluation, and an individual evaluation. Within the individual evaluation, there are stored the operator ID, an operation date and time, an event, and an evaluation value. For example, in the overall evaluation and the individual evaluation, the evaluation value can assume a value that ranges from 0 points (lowest value) to 100 points (highest value).

Storage of the evaluation value within the operation information D4c, for example, is carried out in the following manner. More specifically, an evaluation value is calculated from execution or performance of an operation that is observed from a life log (a video or an action). In addition, an average result (50 points), the result of a skilled worker (100 points), and a failed operation (0 points) are treated beforehand as samples, and are managed by the production management server 12. Further, the observed execution of an operation is compared with the samples in the production management server 12, and the “evaluation value” is calculated based on the similarity thereof with the samples, and is stored in the operation information D4c. Moreover, “execution” of an operation which is specified as a notable log may be added together with the evaluation value to a new sample (a successful operation or a failed operation).

For example, while referring to the operation information D4c that is read out from the operation information DB 74, the life log specifying unit 60 specifies as notable logs, i.e., “successful know-how” life logs D1 to which there are assigned evaluation results having an evaluation value higher than a first threshold value (for example, 80 points). On the other hand, the life log specifying unit 60 specifies as notable logs, i.e., “failure know-how” life logs D1 to which there are assigned evaluation results having an evaluation value lower than a second threshold value (for example, 30 points).

In this manner, the know-how information processing system 10 may be equipped with the evaluation assignment unit 62 configured to assign the evaluation result of success or failure to the action or the event, and the life log specifying unit 60 configured to specify as a notable log a life log D1 whose assigned evaluation result has a degree of success or failure that is greater than the threshold value. Consequently, from among a large number of the life logs D1, it is possible to appropriately extract the notable log to which there is assigned a certain evaluation to the effect of being indicative of successful know-how or failure know-how.

### [Supplemental Considerations]

The present invention is not limited to the embodiment and modifications described above, and it goes without saying that the present invention can be freely modified within a range that does not depart from the essence and gist of the present invention. Alternatively, the respective configurations may be combined arbitrarily within a range in which no technical inconsistencies occur.

For example, in the above-described embodiment and modifications, the know-how information processing system has been described in conformance with an example in which the processing system is applied to agriculture, but the type of industry to which the invention is applicable is not limited to this example. For example, the present invention can be applied to a wide range of industries including forestry, fisheries, manufacturing (in particular, industrial arts manufacturing), the service industry (e.g., restaurants), and the sports industry.

### DESCRIPTION OF REFERENCE CHARACTERS

10 . . . know-how information processing system
12 . . . production management server
14 . . . data center
16, 80 . . . wearable computer
18 . . . environmental sensor group
32 . . . action information acquisition unit
34 . . . biological information acquisition unit
36 . . . know-how information output unit
38 . . . environmental information acquisition unit
56 . . . event information acquisition unit
58 . . . emotional information acquisition unit
60 . . . life log specifying unit
62 . . . evaluation assignment unit
64 . . . know-how information editing unit
71 . . . life log DB (life log recording unit)
72 . . . environmental information DB
73 . . . know-how information DB
74 . . . operation information DB
82 . . . virtual world
D1 . . . life log
D2 . . . environmental information
D3 . . . know-how data (know-how information)
D4 (a to c) . . . operation information
D5 . . . know-how content (know-how information)
NW1, NW2 . . . network
OP1 . . . operator (first target person)
OP2 . . . operator (second target person)

1. A know-how information processing system, comprising:
an action information acquisition unit configured to acquire action information indicative of an action of a target person;
an event information acquisition unit configured to acquire event information related to an event that occurs in vicinity of the target person or an event involving the target person;
a life log recording unit configured to associate the action information acquired by the action information acquisition unit and the event information acquired by the event information acquisition unit, which are acquired respectively at the same time or within a predetermined time period, and record associated information as a life log of the target person;
an environmental information acquisition unit configured to acquire environmental information indicative of a surrounding environment in the vicinity of the target person in which the action or the event occurs; and
a know-how information output unit configured to output as know-how information associated information in which the life log recorded by the life log recording unit is associated with the environmental information acquired by the environmental information acquisition unit, or information generated from the associated information.

2. The know-how information processing system according to claim 1, further comprising:
a biological information acquisition unit configured to acquire biological information indicative of a biological activity of the target person; and
an emotional information acquisition unit configured to acquire emotional information indicative of an emotional state of the target person on a basis of the biological information acquired by the biological information acquisition unit;
wherein the life log recording unit is configured to associate the action information acquired by the action information acquisition unit, the event information acquired by the event information acquisition unit, and the emotional information acquired by the emotional information acquisition unit, which are acquired respectively at the same time or within the predetermined time period, and record associated information as the life log.

3. The know-how information processing system according to claim 2, further comprising:
a life log specifying unit configured to specify as a notable log a life log in which a change in the emotional information is greater than or equal to a threshold value;
wherein the know-how information output unit is configured to output the know-how information including the notable log specified by the life log specifying unit.

4. The know-how information processing system according to claim 1, further comprising:
a life log specifying unit configured to specify as a notable log, from among life logs recorded in past by the life log recording unit and which are associated with the events that are identical or similar to each other, a life log in which a degree of statistical divergence of the action information is greater than or equal to a threshold value;
wherein the know-how information output unit is configured to output the know-how information including the notable log specified by the life log specifying unit.

5. The know-how information processing system according to claim 1, further comprising:
an evaluation assignment unit configured to assign an evaluation result of success or failure to the action or the event; and
a life log specifying unit configured to specify as a notable log a life log whose evaluation result assigned by the evaluation assignment unit has a degree of success or failure that is greater than a threshold value;
wherein the know-how information output unit is configured to output the know-how information including the notable log specified by the life log specifying unit.

6. The know-how information processing system according to claim 3, wherein the know-how information output unit is configured in a manner to generate a virtual world in which the action is virtually experienced using at least the notable log.

7. The know-how information processing system according to claim 1, wherein:
the life log recording unit records life logs for respective target persons; and
the know-how information output unit outputs the know-how information provided by a first target person to a second target person that differs from the first target person, and who has provided a life log having a combination of an event and a surrounding environment which is identical or similar to a combination of the event and the surrounding environment indicated by the know-how information.

8. A know-how information processing method executed by one or a plurality of computers, the method comprising:
an action information acquisition step of acquiring action information indicative of an action of a target person;
an event information acquisition step of acquiring event information related to an event that occurs in vicinity of the target person or an event involving the target person;
a life log recording step of associating the action information and the event information, which are acquired respectively at same time or within a predetermined time period, and recording associated information as a life log of the target person;
an environmental information acquisition step of acquiring environmental information indicative of a surrounding environment in the vicinity of the target person in which the action or the event occurs; and
a know-how information outputting step of outputting as know-how information associated information in which the recorded life log is associated with the acquired environmental information, or information generated from the associated information.

9. A know-how information processing device in which at least a first computer, a second computer, and an environmental sensor group are connected via a network;
wherein the first computer is configured to associate action information indicative of an action of the target person, and event information related to an event that occurs in vicinity of the target person or an event involving the target person, and record associated information as a life log of a target person;
the environmental sensor group is configured to acquire environmental information indicative of a surrounding environment in the vicinity of the target person in which the action or the event occurs; and
the second computer is configured to output as know-how information associated information in which the life log recorded by the first computer is associated with the environmental information acquired by the environmental sensor group, or information generated from the associated information.
